(19) United States
(12) Patent Application Publication
(10) Pub. No.: US 2025/0010036 A1
(43) Pub. Date: Jan. 9, 2025

(54) FLEXIBLE GUIDING SHEATH AND GUIDING DEVICE

(71) Applicant: COPPER MEDICAL TECHNOLOGY CO., LTD., Shenzhen (CN)

(72) Inventors: Dianbiao ZHANG, Shenzhen (CN); Yanfa MAO, Shenzhen (CN); Yaxiong HAN, Shenzhen (CN)

(73) Assignee: COPPER MEDICAL TECHNOLOGY CO., LTD., Shenzhen (CN)

(21) Appl. No.: 18/892,816

(22) Filed: Sep. 23, 2024

Related U.S. Application Data
(63) Continuation of application No. PCT/CN2023/071899, filed on Jan. 12, 2023.

(30) Foreign Application Priority Data
Jul. 5, 2022 (CN) 202210783829.8

(51) Int. Cl. A61M 25/01

(52) U.S. Cl. CPC A61M 25/0141

(57) ABSTRACT

Disclosed are a flexible guiding sheath and a guiding device. A intermediate tube section of the flexible guiding sheath is connected between a bendable tube section and a sheath joint; the bendable tube section includes at least two mortise and tenon connectors which are inserted in sequence along an axial direction of the intermediate tube section; the bendable tube section is configured to, when being applied by a lateral force, be bent axially relative to the intermediate tube section by a relative sliding between the mortise and tenon connector. The bendable tube section of the present disclosure is set to be at least two mortise and tenon connectors which are inserted along the axial direction of the intermediate tube section, such that the flexible guiding sheath can approach the stone fragments in the kidney, and the stone fragments can be flushed out through the injected water flow during the surgery.

### CROSS-REFERENCE TO RELATED APPLICATIONS

This application is a continuation of International Application No. PCT/CN2023/071899, filed on Jan. 12, 2023, which claims priority to Chinese Patent Application No. 202210783829.8, filed on Jul. 5, 2022. All of the aforementioned applications are incorporated herein by reference in their entireties.

### TECHNICAL FIELD

The present disclosure relates to the field of medical technology, and more particularly, to a flexible guiding sheath and a guiding device.

### BACKGROUND

Ureteroscopy lithotripsy is a procedure using a thin tube with a diameter of about 3 mm for insertion into the ureter through the urethra and bladder to break up and remove ureteral stones or kidney stones. A guiding sheath is a commonly-used consumable in the ureteroscopic surgery to establish a direct passage from in vitro environment to the renal pelvis through the urethra and the ureter. A flexible ureteroscope is guided directly into the surgical area by the guiding sheath, allowing for convenient related operations without being affected by the curvature or thickness of the ureter and urethra.

At present, a main body of the guiding sheath used in the ureteroscopic surgery is a relatively flexible tube having a pressure resistance and an anti-twisting performance, such that after the guiding sheath is placed into the human body along a guide wire, a bent part of the ureter can be relatively straightened through the flexibility of the guiding sheath, thereby facilitating the entry and exit, rotation, and other operations of the endoscope. The inventor realized that, due to the flexibility of the guiding sheath, the above guiding sheath can only be placed on the upper section of the ureter or at a position just entering the renal pelvis, forming a passage parallel to the longitudinal axis of the human body; however, the guiding sheath cannot be bent to reach a deeper position inside the renal pelvis or the calyces, thus, it is difficult for the guiding sheath to approach the crushed stones during the ureteroscopic surgery, and the stone fragments cannot be flushed out from the guiding sheath through the injected water flow during the ureteroscopic surgery. Therefore, the efficiency of removing stone fragments in the ureteroscopic surgery is low.

### SUMMARY

The present disclosure provides a flexible guiding sheath and a guiding device to solve the technical problems that the guiding sheath in the existing technology has a high toughness and cannot be bent.

In view of the above technical problems, an embodiment of the present disclosure provides a flexible guiding sheath; the flexible guiding sheath includes a bendable tube section, an intermediate tube section, and a sheath joint, the intermediate tube section is connected between the bendable tube section and the sheath joint; the bendable tube section comprises at least two mortise and tenon connectors which are inserted in sequence along an axial direction of the intermediate tube section; the bendable tube section is configured to, when being applied by a lateral force, be bent axially relative to the intermediate tube section by a relative sliding between the mortise and tenon connector.

Another embodiment of the present disclosure provides a guiding device, including a dilator and the aforementioned flexible guiding sheath, wherein the dilator is connected to the sheath joint through the flexible guiding sheath.

The flexible guiding sheath of the present disclosure includes three parts: the bendable tube section, the intermediate tube section, and the sheath joint. By arranging the bendable tube section with at least two mortise and tenon connectors that are inserted sequentially along the axial direction of the intermediate tube section, the flexible guiding sheath can be bent into the desired shape, allowing the flexible guiding sheath to approach the stone fragments in the kidney through the bendable tube section, and then to flush out the stone fragments from the flexible guiding sheath through the injected water flow during the surgery, which improves the efficiency of removing the stone fragments, avoids the situations affecting the efficiency of the surgery caused by the impact of stone powder on the field of vision or accumulation in the kidney, avoids the uncertainty of natural stone removal, and improves the stone removal effect.

The details of one or more embodiments of the present disclosure are presented in the following figures and descriptions. Other features and advantages of the present disclosure will become apparent from the specification, drawings, and claims.

### BRIEF DESCRIPTION OF THE DRAWINGS

To describe the technical solutions in the embodiments of the present disclosure more clearly, the following briefly introduces the accompanying drawings required for describing the embodiments.

FIG. 1 is a schematic view of a flexible guiding sheath according to an embodiment of the present disclosure.

FIG. 2 is a schematic view of a flexible guiding sheath according to another embodiment of the present disclosure.

FIG. 3 is a schematic view of a mortise and tenon connector of the flexible guiding sheath in FIG. 2 of the present disclosure.

FIG. 4 is a schematic view of a flexible guiding sheath according to another embodiment of the present disclosure.

FIG. 5 is a structural view of the mortise and tenon connector of the flexible guiding sheath in FIG. 4 of the present disclosure.

FIG. 6 is a schematic view of a flexible guiding sheath according to another embodiment of the present disclosure.

FIG. 7 is a schematic view of the mortise and tenon connector of the flexible guiding sheath in FIG. 6 of the present disclosure.

FIG. 8 is a schematic view of a guiding device according to an embodiment of the present disclosure.

### DESCRIPTION OF REFERENCE NUMERALS

1, bendable tube section; 2, intermediate tube section; 3, sheath joint; 4, dilator; 11, mortise and tenon connector; 12, elastic material layer; 1101, first body; 1102, first inserting portion; 1103, second inserting portion; 1104, first insertion sliding slot; 1105, second insertion sliding slot; 11021, first plug; 11022, first connecting section; 11041, first limiting opening; 11031, second plug; 11032, second connecting section; 11051, second limiting opening; 11023, first arc surface; 11033, second arc surface; 11052, second arc slot; 11042, first arc slot; 1106, second body; 1107, first rotating slot; 1108, first stop block; 1109, first rotating block; 11071, first opening; 1110, second stop block; 1111, second rotating block; 1112, second rotating slot; 11121, second opening; 1113, third stop block; 1114, third rotating block; 1115, third rotating slot; 11151, third opening; 1116, first clamping slot; 1117, second clamping slot; 1118, first buckle; 1119, second buckle; 22, first protective layer; 23, support structure layer; 24, second protective layer.

### DETAILED DESCRIPTION OF THE EMBODIMENTS

In order to make the objectives, features, and advantages of the present disclosure more obvious and understandable, the technical solutions in the embodiments of the present disclosure will be clearly and completely described below in conjunction with the accompanying drawings of the embodiments of the present disclosure. It is apparent that the embodiments described below are only a part of the embodiments of the present disclosure, not all of them. Based on the embodiments of the present disclosure, all other embodiments obtained by those skilled in the art without creative effort fall within the scope of protection of the present disclosure.

It should be understood that the terms “up”, “down”, “left”, “right”, “front”, “back”, “middle”, etc. indicate the orientation or positional relationship based on the orientation or positional relationship shown in the drawings, which is only for the convenience of describing the present disclosure and simplifying the description, rather than indicating or implying that the device or element referred to must have a specific orientation, be constructed and operated in a specific orientation, and therefore cannot be understood as a limitation of the present disclosure.

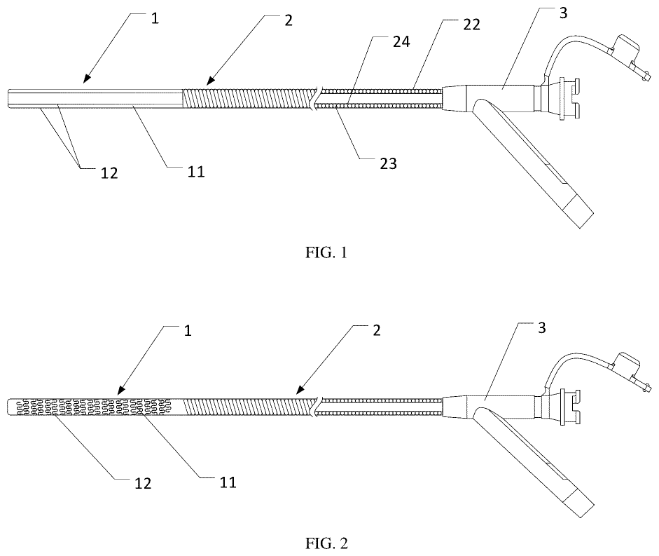

As shown in FIG. 1, an embodiment of the present disclosure provides a flexible guiding sheath, which includes a bendable tube section 1, an intermediate tube section 2, and a sheath joint 3. The intermediate tube section 2 is connected between the bendable tube section 1 and the sheath joint 3. The bendable tube section 1 includes at least two mortise and tenon connectors 11 which are inserted in sequence along an axial direction of the intermediate tube section 2. When being applied by a lateral force, the bendable tube section 1 is bent axially relative to the intermediate tube section 2 through the relative sliding between the mortise and tenon connectors 11. In one embodiment, on the basis that the connection between the intermediate tube section 2, the bendable tube section 1, and the sheath joint 3 can be stabilized, the connection method between the intermediate tube section 2, the bendable tube section 1, and the sheath joint 3 can be, but is not limited to one or more of the following connection methods: integral molding, bonding, welding, snap connection, an threaded connection.

In one embodiment, as shown in FIG. 1, the intermediate tube section 2 includes a first protective layer 22, a support structure layer 23, and a second protective layer 24. The first protective layer 22 encloses an outer wall of the support structure layer 23, and the second protective layer 24 encloses an inner wall of the support structure layer 23. The support structure layer 23 is a spring layer, the first protective layer 22 is made of PEBAX (block polyetheramide resin), and the second protective layer 24 is made of PTFE (polytetrafluoroethylene). It can be understood that the first protective layer 22 and the second protective layer 24 respectively enclose an outer surface and an inner surface of the support structure layer 23 to protect the support structure layer 23. In one embodiment, a lubricating coating is formed on the surface of the first protective layer 22 and the surface of the second protective layer 24 in contact with the external environment. The lubricating coating formed on the first protective layer 22 which encloses the outer surface of the support structure layer 23 is used to facilitate a smoother insertion of the intermediate tube section 2 into the ureter and a removal of the intermediate tube section 2 from the ureter. The lubricating coating formed on the second protective layer 24 which encloses the inner surface of the support structure layer 23 is used to facilitate a smoother insertion of a soft lens or another instrument into the intermediate tube section 2 and a removal of the soft lens or the instrument from the intermediate tube section 2.

In the embodiment of the present disclosure, the flexible guiding sheath includes three parts: the bendable tube section 1, the intermediate tube section 2, and the sheath joint 3. In the above embodiment, the support structure layer 23 of the intermediate tube section 2 is a spring layer, which enables the intermediate tube section 2 to maintain a toughness similar to the guiding sheath in the existing technology. However, based on the guiding sheath in the existing technology, the flexible guiding sheath of the present disclosure further includes the bendable tube section 1. By arranging the bendable tube section 1 with at least two mortise and tenon connectors 11 which are inserted in sequence along the axial direction of the intermediate tube section 2, the flexible guiding sheath can be bent into a desired shape, such that the flexible guiding sheath can approach the stone fragments in the kidney through the bendable tube section 1, and then flush out the stone fragments from the flexible guiding sheath through the injected water flow during the surgery. In this way, the efficiency of removing the stone fragments is improved, and the situation that the efficiency of the surgery is affected by the impact of stone powder on the field of vision or accumulation in the kidney can be avoided. Furthermore, the uncertainty of natural stone removal can be avoided to improve the stone removal effect.

In one embodiment, as shown in FIG. 1, the bendable tube section 1 further includes an elastic material layer 12 coated on a surface of the mortise and tenon connector 11. It can be understood that the elastic material layer 12 is used to restrict a radial movement of the mortise and tenon connector 11 in the bendable tube section 1, which binds the mortise and tenon connector 11 to prevent components of the mortise and tenon connector 11 from separating from each other; furthermore, the elastic material layer 12 can be deformed when the bendable tube section 1 is bent to keep enclosing the surface of the mortise and tenon connector 11. The elastic material layer 12 can be coated on an outer surface and/or an inner surface of the mortise and tenon connector 11 to avoid interference caused by the direct contact between the mortise and tenon connector 11 and the external environment. In one embodiment, a lubricating coating is formed on an innermost layer and an outermost layer of the bendable tube section 1, allowing for a smoother insertion of the bendable tube section 1 or a smoother insertion into the bendable tube section 1.

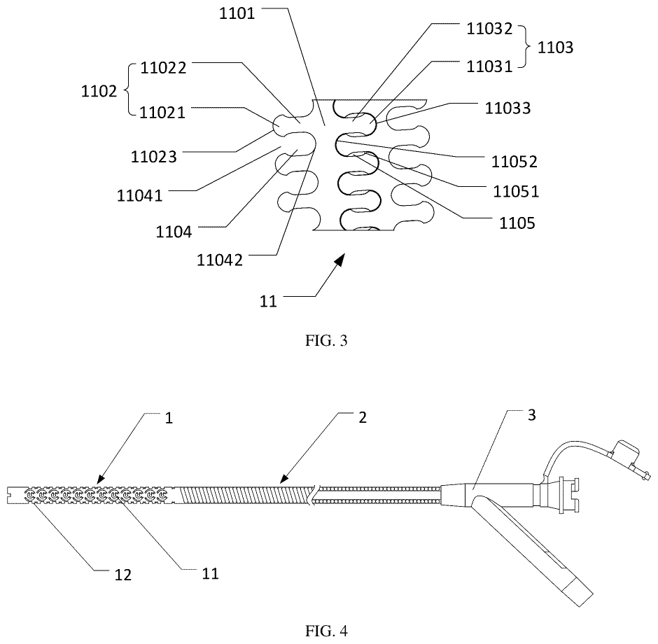

In an embodiment, as shown in FIGS. 2 and 3, each of the mortise and tenon connectors 11 includes a first body 1101, at least two first inserting portions 1102, and at least two second inserting portions 1103. The first inserting portions 1102 and the second inserting portions 1103 are symmetrically connected to opposite sides of the first body 1101. A first insertion sliding slot 1104 is formed between two first inserting portions 1102 allowing for the insertion and relative sliding of the second inserting portion 1103 of the adjacent mortise and tenon connector 11. A second insertion sliding slot 1105 is formed between two second inserting portions 1103 allowing for the insertion and relative sliding of the first inserting portion 1102 of the adjacent mortise and tenon connector 11. It can be understood that, on the basis that the bendable tube section 1 can be bent when the mortise and tenon connector 11 is stretched and/or contracted, the first body 1101 can have an annular structure, a sheet structure, or other shapes. In an embodiment, the first body 1101 has an annular structure, the first inserting portions 1102 and the first insertion sliding slots 1104 are distributed on one side of the first body 1101, and the second inserting portions 1103 and the second insertion sliding slots 1105 are distributed on the other side of the first body 1101. The first inserting portion 1102 is inserted into the corresponding second insertion sliding slot 1105 and can slide in the corresponding second insertion sliding slot 1105, and the second inserting portion 1103 is inserted into the corresponding first insertion sliding slot 1104 and can slide in the corresponding first insertion sliding slot 1104, thereby forming a bendable tubular structure between the two mortise and tenon connectors 11 through the above sliding connection relationship.

In an embodiment, as shown in FIGS. 2 and 3, each of the first inserting portions 1102 includes a first plug 11021 and a first connecting section 11022 connected between the first body 1101 and the first plug 11021. Each of the second inserting portions 1103 includes a second plug 11031 and a second connecting section 11032 connected between the first body 1101 and the second plug 11031. A first limiting opening 11041 is formed in the first insertion sliding slot 1104 allowing for the second connecting section 11032 to pass through the first limiting opening 11041, and a second limiting opening 11051 is formed in the second insertion sliding slot 1105 allowing for the first connecting section 11022 to pass through the second limiting opening 11051. In a direction perpendicular to an axial direction of the bendable tube section 1 in an unbent state, a maximum width of the first plug 11021 is greater than a width of the second limiting opening 11051; when the bendable tube section 1 is bent, the first plug 11021 slides along the second insertion sliding slot 1105; a maximum width of the second plug 11031 is greater than a width of the first limiting opening 11041; when the bendable tube section 1 is bent, the second plug 11031 slides along the first insertion sliding slot 1104. It can be understood that, in the direction perpendicular to the axial direction of the bendable tube section 1 when the bendable tube section is unbent, the maximum width of the first plug 11021 is greater than the width of the second limiting opening 11051, and the second limiting opening 11051 restricts the first inserting portion 1102 from disengaging from the second insertion sliding slot 1105; the maximum width of the second plug 11031 is greater than the width of the first limiting opening 11041, and the first limiting opening 11041 restricts the second inserting portion 1103 from disengaging from the first insertion sliding slot 1104. It can be understood that in the direction perpendicular to the axial direction of the bendable tube section 1 when the bendable tube section 1 is unbent, the maximum width of the first plug 11021 is less than or equal to the width of the second insertion sliding slot 1105, such that the first plug 11021 can slide within the second insertion sliding slot 1105; a maximum width of the second plug 11031 is less than or equal to a width of the first insertion sliding slot 1104, such that the second plug 11031 can slide within the first insertion sliding slot 1104. It can be understood that in the direction perpendicular to the axial direction of the bendable tube section 1 when the bendable tube section 1 is unbent, the maximum width of the first connecting section 11022 is less than the width of the second limiting opening 11051, such that when the first plug 11021 slides along the second insertion sliding slot 1105, the first connecting section 11022 slides within the second limiting opening 11051; the maximum width of the second connecting section 11032 is less than the width of the first limiting opening 11041, such that when the second plug 11031 slides along the first insertion sliding slot 1104, the second connecting section 11032 slides within the first limiting opening 11041.

In an embodiment, as shown in FIG. 2 and FIG. 3, a first arc surface 11023 is formed on one end of the first plug 11021 away from the first body 1101, a second arc surface 11033 is formed on one end of the second plug 11031 away from the first body 1101, a second arc slot 11052 engageable with the first arc surface 11023 is formed in one end of the second insertion sliding slot 1105 close to the first body 1101, and a first arc slot 11042 engageable with the second arc surface 11033 is formed in one end of the first insertion sliding slot 1104 close to the first body 1101. The second limiting opening 11051 is formed between the two second plugs 11031, the first plug 11021 can rotate in the second insertion sliding slot 1105, the first limiting opening 11041 is formed between the two first plugs 11021, and the second plug 11031 can rotate in the first insertion sliding slot 1104.

In the embodiments of the present disclosure, at least two annular mortise and tenon connectors 11 engaging with each other are provided, and the mortise and tenon connector 11 has a semi-circular concave-convex connection structure, realizing the universal bending of the bendable tube section 1 and allowing the bendable tube section 1 to expand and contract within a certain limit, such that a sheath opening of the flexible guiding sheath can more easily approach the direction of the kidney stones.

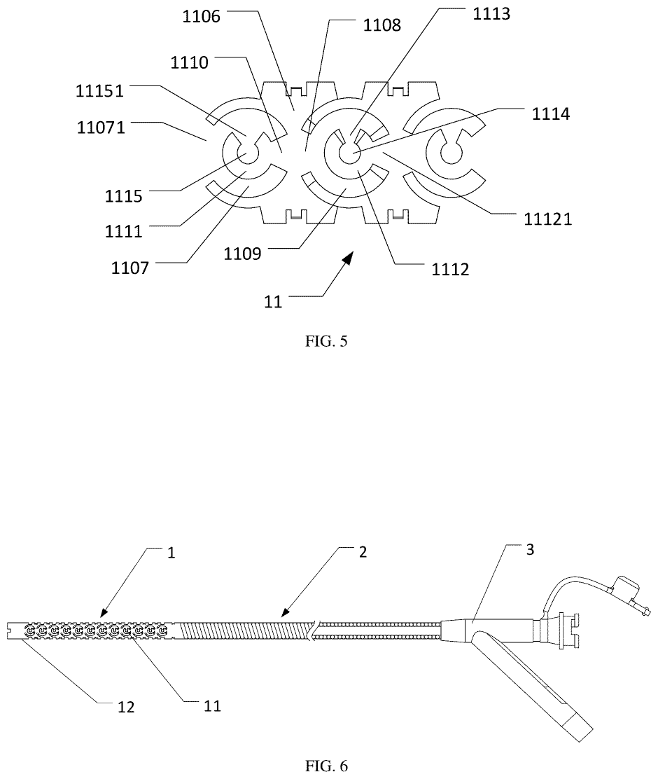

In one embodiment, as shown in FIGS. 4 and 5, each of the mortise and tenon connectors 11 includes a second body 1106, a first rotating slot 1107 formed in a first side of the second body 1106, a first stop block 1108 connected to a second side of the second body 1106, and a first rotating block 1109 connected to a second side of the first stop block 1108. A first opening 11071 is formed in the first rotating slot 1107 for allowing the first stop block 1108 of the adjacent mortise and tenon connector 11 to pass through first opening 11071. The first rotating slot 1107 is used to accommodate the first rotating block 1109 of the adjacent mortise and tenon connector 11. A maximum width of the first rotating block 1109 is greater than a width of the first opening 11071. When the bendable tube section 1 is bent, the first rotating block 1109 rotates relatively to the first rotating slot 1107, and when the first rotating blocks 1109 of the two adjacent mortise and tenon connectors 11 are abutted by an edge of the first opening 11071, the two adjacent mortise and tenon connectors 11 stop rotating. In one embodiment, the maximum width of the first rotating block 1109 is greater than the width of the first opening 11071, thus, the first rotating block 1109 can be inserted into the first rotating slot 1107 to rotate without detaching from the first rotating slot 1107, thereby allowing the first rotating block 1109 to rotate in the first rotating slot 1107. The first stop block 1108 is used to limit a rotation angle between the two mortise and tenon connectors 11.

In one embodiment, as shown in FIGS. 4 and 5, each of the mortise and tenon connectors 11 further includes a second stop block 1110 connected to an inner wall of the first rotating slot 1107, and a second rotating block 1111 connected to a first side of the second stop block 1110 and located in the first rotating slot 1107. A second rotating slot 1112 is formed in the first rotating block 1109. A second opening 11121 is formed in the second rotating slot 1112 facing away from the first opening 11071 and used for allowing the second stop block 1110 of the adjacent mortise and tenon connector 11 to pass through the second opening 11121. The second rotating slot 1112 is used to accommodate the second rotating block 1111 of the adjacent mortise and tenon connector 11. A maximum width of the second rotating block 1111 is greater than a width of the second opening 11121. When the bendable tube section 1 is bent, the first rotating block 1109 rotates relatively to the first rotating slot 1107, and the second rotating block 1111 rotates relatively to the second rotating slot 1112. When the first rotating blocks 1109 of the two adjacent mortise and tenon connectors 11 abut the edge of the first opening 11071, or the second rotating blocks 1111 of the two adjacent mortise and tenon connectors 11 abut an edge of the second opening 11121, the two adjacent mortise and tenon connectors 11 stop rotating. In one embodiment, the maximum width of the second rotating block 1111 is greater than the width of the second opening 11121, thus, the second rotating block 1111 can be inserted into the second rotating slot 1112 to rotate without detaching from the second rotating slot 1112. Cross-sections of the first rotating slot 1107, the second rotating slot 1112, the first rotating block 1109, and the second rotating block 1111 are all arc. The cross-section of the first rotating block 1109 is engageable with that of the first rotating slot 1107, and the cross-section of the second rotating slot 1112 is engageable with that of the second rotating slot 1112. Therefore, while the first rotating block 1109 rotates within the first rotating slot 1107, the second rotating block 1111 can rotate within the second rotating slot 1112. The second stop block 1110 is used to limit the rotation angle between the two mortise and tenon connectors 11.

In one embodiment, as shown in FIGS. 4 and 5, each of the mortise and tenon connectors 11 further includes a third stop block 1113 connected to an inner wall of the second rotating slot 1112, and a third rotating block 1114 connected to the third stop block 1113 and located in the second rotating slot 1112. A third rotating slot 1115 is formed in the second rotating block 1111. A third opening 11151 is formed in the third rotating slot 1115 allowing for the third stop block 1113 of the adjacent mortise and tenon connector 11 to pass through the third opening 11151. The third rotating slot 1115 is used to accommodate the third rotating block 1114 of the adjacent mortise and tenon connector 11. A maximum width of the third rotating block 1114 is greater than a width of the third opening 11151. When the bendable tube section 1 is bent, the first rotating block 1109 rotates relatively to the first rotating slot 1107, the second rotating block 1111 rotates relatively to the second rotating slot 1112, and the third rotating block 1114 rotates relatively to the third rotating slot 1115. When the first rotating blocks 1109 of the two adjacent mortise and tenon connectors 11 abut the edge of the first opening 11071, the second rotating blocks 1111 of the two adjacent mortise and tenon connectors 11 abut the edge of the second opening 11121, or when the third rotating blocks 1114 of the two adjacent mortise and tenon connectors 11 abut an edge of the third opening 11151, the two adjacent mortise and tenon connectors 11 stop rotating. In one embodiment, the maximum width of the third rotating block 1114 is greater than the width of the third opening 11151, thus, the third rotating block 1114 can be inserted into the third rotating slot 1115 to rotate without detaching from the third rotating slot 1115. A cross-section of the third rotating slot 1115 is circular, and a cross-section of the third rotating block 1114 is circular and is engageable with the cross-section of the third rotating slot 1115, therefore, while the first rotating block 1109 rotates in the first rotating slot 1107, the second rotating block 1111 can rotate in the second rotating slot 1112, and the third rotating block 1114 can rotate in the third rotating slot 1115. The third stop block 1113 is used to limit the rotation angle between the two mortise and tenon connectors 11.

In one embodiment, as shown in FIGS. 4 and 5, a preset angle is formed between an opening direction of the third opening 11151 and an opening direction of the second opening 11121. It can be understood that the preset angle between the opening directions of the third opening 11151 and the second opening 11121 ranges from 0 to 180 degrees. In an embodiment, the preset angle between the opening directions of the third opening 11151 and the second opening 11121 is 90 degrees.

In the embodiment of the present disclosure, at least two mortise and tenon connectors 11 engaging with each other are provided, and the mortise and tenon connector 11 has a circular concave-convex connection structure to increase the bending angle of the bendable tube section 1, such that the sheath opening of the flexible guiding sheath can approach towards the direction of the kidney stones more easily.

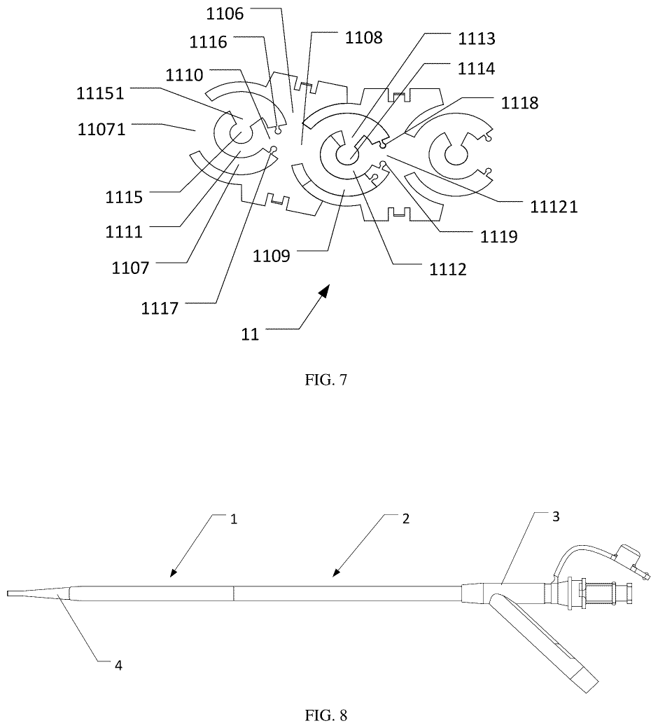

In one embodiment, as shown in FIGS. 6 and 7, the mortise and tenon connector 11 further includes a first clamping slot 1116 and a second clamping slot 1117 respectively formed in opposite sides of the second block 1110; a first buckle 1118 being engageable with the first clamping slot 1116 and a second buckle 1119 being engageable with the second clamping slot 1117 are respectively arranged in opposite sides of the second opening 11121. When the bendable tube section 1 is bent, the first rotating block 1109 rotates relatively to the first rotating slot 1107, and the second rotating block 1111 rotates relatively to the second rotating slot 1112; when the second blocks 1110 of the two mortise and tenon connectors 11 abut the edge of the second opening 11121, the two adjacent mortise and tenon connectors 11 stop rotating; when the first buckle 1118 engages with the first clamping slot 1116 or the second buckle 1119 engages with the second clamping slot 1117, the relative rotational position between the two adjacent mortise and tenon connectors 11 is fixed. In one embodiment, the first buckle 1118 and the second buckle 1119 are made of flexible material, and the first clamping slot 1116 and the second clamping slot 1117 are made of hard material. Therefore, the first buckle 1118 can be inserted into the first clamping slot 1116 and detached from the first clamping slot 1116 under a certain force, and the second buckle 1119 can be inserted into the second clamping slot 1117 and detached from the second clamping slot 1117 under a certain force. It can be understood that, on the basis that the first buckle 1118 can be inserted into the first clamping slot 1116 and detached from the first clamping slot 1116 under a certain force, and the second buckle 1119 can be inserted into the second clamping slot 1117 and detached from the second clamping slot 1117 under a certain force, the first buckle 1118 and the second buckle 1119 can also be made of hard and/or flexible material, and the first clamping slot 1116 and the second clamping slot 1117 can also be made of flexible and/or hard material. In one embodiment, the first clamping slot 1116 and the second clamping slot 1117 can also be formed in opposite sides of the third stop block 1113. At the same time, the first buckle 1118 and the second buckle 1119 can also be arranged on opposite sides of the third opening 11151. It can be understood that the first clamping slot 1116 and the second clamping slot 1117 can be formed in an abutting surface of one mortise and tenon connector 11, and the first buckle 1118 and the second buckle 1119 can be arranged on the abutting surface of the other mortise and tenon connector 11, such that the two adjacent mortise and tenon connectors 11 can be fixed at a preset rotational position when relatively sliding to each other.

In the embodiments of the present disclosure, with the slot (such as the first clamping slot 1116 and the second clamping slot 1117) and the buckle (such as the first buckle 1118 and the second buckle 1119) of the mortise and tenon connector 11 having a circular concave-convex connection structure, when the bendable tube section 1 is bent to a preset angle, the bendable tube section 1 is in a fixed-angle state through the engagement between the slot and the buckle. After the bendable endoscope in the flexible guiding sheath is removed, the bendable tube section 1 remains in the fixed-angle state, thereby facilitating the next step of negative pressure suction of stone fragments. When the bendable tube section 1 is required to release from the fixed-angle state, the endoscope is bent reversely to drive the bendable tube section 1 to bend reversely, allowing the slot to disengage from the buckle and releasing the bendable tube section 1 from the fixed-angle state.

As shown in FIG. 8, an embodiment of the present disclosure further provides a guiding device, including a dilator 4 and the above-mentioned flexible guiding sheath. The dilator 4 passes through the sheath joint 3 to be connected to the sheath joint 3. In an embodiment, the dilator 4 is connected to the sheath joint 3 by passing through the bendable tube section 1, the intermediate tube section 2, and the sheath joint 3 of the flexible guiding sheath in turn.

In the embodiments of the present disclosure, the guiding device includes the flexible guiding sheath and the dilator 4 that passes through the flexible guiding sheath and is connected to the sheath joint 3 of the flexible guiding sheath. The flexible guiding sheath includes three parts: the bendable tube section 1, the intermediate tube section 2, and the sheath joint 3. By arranging the bendable tube section 1 with at least two mortise and tenon connectors 11 that are inserted sequentially along the axial direction of the intermediate tube section 2, the guiding device can be bent into a desired shape, such that the guiding device can approach the stone fragments in the kidney through the bendable tube section 1, allowing the stone fragments to be flushed out of the guiding device through the injected water flow during the surgery. In this way, the efficiency of removing the stone fragments is improved, and the situation that the efficiency of the surgery is affected by the impact of stone powder on the field of vision or accumulation in the kidney can be avoided; furthermore, the uncertainty of natural stone removal can be avoided to improve the stone removal effect.

The above descriptions are only the embodiments of the flexible guiding sheath and the guiding device of the present disclosure, which do not limit the scope of the present disclosure. Any equivalent structure or equivalent process transformation made by using the content of the description and drawings of the present disclosure, or directly or indirectly applied to other related technologies in the same way, all fields are included in the scope of patent protection of the present disclosure.

What is claimed is:

1. A flexible guiding sheath, comprising a bendable tube section, an intermediate tube section, and a sheath joint, wherein
the intermediate tube section is connected between the bendable tube section and the sheath joint;
the bendable tube section comprises at least two mortise and tenon connectors which are inserted in sequence along an axial direction of the intermediate tube section; and
the bendable tube section is configured to, when being applied by a lateral force, be bent axially relative to the intermediate tube section by a relative sliding between the mortise and tenon connector.

2. The flexible guiding sheath according to claim 1, wherein the bendable tube section further comprises an elastic material layer coated on a surface of the mortise and tenon connector.

3. The flexible guiding sheath according to claim 1, wherein the mortise and tenon connector comprises a first body, at least two first inserting portions, and at least two second inserting portions;
the first inserting portions and the second inserting portions are symmetrically connected to opposite sides of the first body;
a first insertion sliding slot in formed between the two first inserting portions for allowing the second inserting portion of the adjacent mortise and tenon connector to be inserted into the first insertion sliding slot and slide relatively to the first insertion sliding slot; and
a second insertion sliding slot is formed between the two second inserting portions for allowing the first inserting portion of the adjacent mortise and tenon connector to be inserted into the second insertion sliding slot and slide relatively to the second insertion sliding slot.

4. The flexible guiding sheath according to claim 3, wherein the first inserting portion comprises a first plug and a first connecting section connected between the first body and the first plug;
the second inserting portion comprises a second plug and a second connecting section connected between the first body and the second plug;
a first limiting opening is formed in the first insertion sliding slot for allowing the second connecting section to pass through the first limiting opening;
a second limiting opening is formed in the second insertion sliding slot for allowing the first connecting section to pass through the second limiting opening;
a maximum width of the first plug is greater than a width of the second limiting opening in a direction perpendicular to an axial direction of the bendable tube section in an unbent state; when the bendable tube section is bent, the first plug slides along the second insertion sliding slot; and
a maximum width of the second plug is greater than a width of the first limiting opening; when the bendable tube section is bent, the second plug slides along the first insertion sliding slot.

5. The flexible guiding sheath according to claim 1, wherein the mortise and tenon connector comprises a second body, a first rotating slot formed in a first side of the second body, a first stop block connected to a second side of the second body, and a first rotating block connected to a second side of the first stop block;
a first opening is formed in the first rotating slot for allowing the first stop block of the adjacent mortise and tenon connector to pass through the first opening, and the first rotating slot is configured to accommodate the first rotating block of the adjacent mortise and tenon connector; a maximum width of the first rotating block is greater than a width of the first opening; and
when the bendable tube section is bent, the first rotating block rotates relatively to the first rotating slot; when the first rotating blocks of the two adjacent mortise and tenon connectors are abutted by an edge of the first opening, the two adjacent mortise and tenon connectors stop rotating.

6. The flexible guiding sheath according to claim 5, wherein the mortise and tenon connector further comprises a second stop block connected to an inner wall of the first rotating slot, and a second rotating block connected to a first side of the second stop block and located in the first rotating slot;
a second rotating slot is formed in the first rotating block, and a second opening facing away from the first opening is formed in the second rotating slot for allowing the second stop block of the adjacent mortise and tenon connector to pass through the second opening; the second rotating slot is configured to accommodate the second rotating block of the adjacent mortise and tenon connector; a maximum width of the second rotating block is greater than a width of the second opening; and
when the bendable tube section is bent, the first rotating block rotates relatively to the first rotating slot, and the second rotating block rotates relatively to the second rotating slot; when the first rotating blocks of the two adjacent mortise and tenon connectors abut the edge of the first opening or the second rotating blocks of the two adjacent mortise and tenon connectors abut an edge of the second opening, the two adjacent mortise and tenon connectors stop rotating.

7. The flexible guiding sheath according to claim 6, wherein the mortise and tenon connector further comprises a first clamping slot and a second clamping slot respectively formed in opposite sides of the second stop block; a first buckle engageable with the first clamping slot and a second buckle engageable with the second clamping slot are respectively arranged on the opposite sides of the second opening;
when the bendable tube section is bent, the first rotating block rotates relatively to the first rotating slot, and the second rotating block rotates relatively to the second rotating slot; and
when the second stop blocks of the two adjacent mortise and tenon connectors abut the edge of the second opening, the two adjacent mortise and tenon connectors stop rotating; when the first buckle engages with the first clamping slot or the second buckle engages with the second clamping slot, a relative rotational position between the two adjacent mortise and tenon connectors is fixed.

8. The flexible guiding sheath according to claim 6, wherein the mortise and tenon connector further comprises a third stop block connected to an inner wall of the second rotating slot, and a third rotating block connected to the third stop block and located in the second rotating slot; a third rotating slot is formed in the second rotating block, and a third opening is formed in the third rotating slot for allowing the third stop block of the adjacent mortise and tenon connector to pass through the third opening; the third rotating slot is configured to accommodate the third rotating block of the adjacent mortise and tenon connector; a maximum width of the third rotating block is greater than a width of the third opening; and
when the bendable tube section is bent, the first rotating block rotates relatively to the first rotating slot, the second rotating block rotates relatively to the second rotating slot, and the third rotating block rotates relatively to the third rotating slot; when the first rotating blocks of the two adjacent mortise and tenon connectors abut the edge of the first opening, the second rotating blocks of the two adjacent mortise and tenon connectors abut the edge of the second opening, or the third rotating blocks of the two adjacent mortise and tenon connectors abut an edge of the third opening, the two adjacent mortise and tenon connectors stop rotating.

9. The flexible guiding sheath according to claim 8, wherein the mortise and tenon connector further comprises a first clamping slot and a second clamping slot respectively formed in opposite sides of the second stop block; a first buckle engageable with the first clamping slot and a second buckle engageable with a second clamping slot are respectively arranged on opposite sides of the second opening;
when the bendable tube section is bent, the first rotating block rotates relatively to the first rotating slot, and the second rotating block rotates relatively to the second rotating slot; and
when the second stop blocks of the two adjacent mortise and tenon connectors abut the edge of the second opening, the two adjacent mortise and tenon connectors stop rotating; when the first buckle engages with the first clamping slot or the second buckle engages with the second clamping slot, a relative rotational position between the two adjacent mortise and tenon connectors is fixed.

10. The flexible guiding sheath according to claim 8, wherein a preset angle is formed between an opening direction of the third opening and an opening direction of the second opening.

11. The flexible guiding sheath according to claim 10, wherein the mortise and tenon connector further comprises a first clamping slot and a second clamping slot respectively formed in opposite sides of the second stop block; a first buckle engageable with the first clamping slot and a second buckle engageable with the second clamping slot are respectively arranged on opposite sides of the second opening;
when the bendable tube section is bent, the first rotating block rotates relatively to the first rotating slot, and the second rotating block rotates relatively to the second rotating slot; and
when the second stop blocks of the two adjacent mortise and tenon connectors abut the edge of the second opening, the two adjacent mortise and tenon connectors stop rotating; when the first buckle engages with the first clamping slot or the second buckle engages with the second clamping slot, a relative rotational position between the two adjacent mortise and tenon connectors is fixed.

12. A guiding device, comprising a dilator and the flexible guiding sheath according to claim 1, wherein the dilator is connected to the sheath joint through the flexible guiding sheath.

13. The guiding device according to claim 12, wherein the bendable tube section further comprises an elastic material layer coated on a surface of the mortise and tenon connector.

14. The guiding device according to claim 12, wherein the mortise and tenon connector comprises a first body, at least two first inserting portions, and at least two second inserting portions;
the first inserting portions and the second inserting portions are symmetrically connected to opposite sides of the first body;
a first insertion sliding slot in formed between the two first inserting portions for allowing the second inserting portion of the adjacent mortise and tenon connector to be inserted into the first insertion sliding slot and slide relatively to the first insertion sliding slot; and
a second insertion sliding slot is formed between the two second inserting portions for allowing the first inserting portion of the adjacent mortise and tenon connector to be inserted into the second insertion sliding slot and slide relatively to the second insertion sliding slot.

15. The guiding device according to claim 14, wherein the first inserting portion comprises a first plug and a first connecting section connected between the first body and the first plug;
the second inserting portion comprises a second plug and a second connecting section connected between the first body and the second plug;
a first limiting opening is formed in the first insertion sliding slot for allowing the second connecting section to pass through the first limiting opening;
a second limiting opening is formed in the second insertion sliding slot for allowing the first connecting section to pass through the second limiting opening;
a maximum width of the first plug is greater than a width of the second limiting opening in a direction perpendicular to an axial direction of the bendable tube section in an unbent state; when the bendable tube section is bent, the first plug slides along the second insertion sliding slot; and
a maximum width of the second plug is greater than a width of the first limiting opening; when the bendable tube section is bent, the second plug slides along the first insertion sliding slot.

16. The guiding device according to claim 12, wherein the mortise and tenon connector comprises a second body, a first rotating slot formed in a first side of the second body, a first stop block connected to a second side of the second body, and a first rotating block connected to a second side of the first stop block;
a first opening is formed in the first rotating slot for allowing the first stop block of the adjacent mortise and tenon connector to pass through the first opening, and the first rotating slot is configured to accommodate the first rotating block of the adjacent mortise and tenon connector; a maximum width of the first rotating block is greater than a width of the first opening; and
when the bendable tube section is bent, the first rotating block rotates relatively to the first rotating slot; when the first rotating blocks of the two adjacent mortise and tenon connectors are abutted by an edge of the first opening, the two adjacent mortise and tenon connectors stop rotating.

17. The guiding device according to claim 16, wherein the mortise and tenon connector further comprises a second stop block connected to an inner wall of the first rotating slot, and a second rotating block connected to a first side of the second stop block and located in the first rotating slot; a second rotating slot is formed in the first rotating block, and a second opening facing away from the first opening is formed in the second rotating slot for allowing the second stop block of the adjacent mortise and tenon connector to pass through the second opening; the second rotating slot is configured to accommodate the second rotating block of the adjacent mortise and tenon connector; a maximum width of the second rotating block is greater than a width of the second opening; and
when the bendable tube section is bent, the first rotating block rotates relatively to the first rotating slot, and the second rotating block rotates relatively to the second rotating slot; when the first rotating blocks of the two adjacent mortise and tenon connectors abut the edge of the first opening or the second rotating blocks of the two adjacent mortise and tenon connectors abut an edge of the second opening, the two adjacent mortise and tenon connectors stop rotating.

18. The guiding device according to claim 17, wherein the mortise and tenon connector further comprises a third stop block connected to an inner wall of the second rotating slot, and a third rotating block connected to the third stop block and located in the second rotating slot; a third rotating slot is formed in the second rotating block, and a third opening is formed in the third rotating slot for allowing the third stop block of the adjacent mortise and tenon connector to pass through the third opening; the third rotating slot is configured to accommodate the third rotating block of the adjacent mortise and tenon connector; a maximum width of the third rotating block is greater than a width of the third opening; and
when the bendable tube section is bent, the first rotating block rotates relatively to the first rotating slot, the second rotating block rotates relatively to the second rotating slot, and the third rotating block rotates relatively to the third rotating slot; when the first rotating blocks of the two adjacent mortise and tenon connectors abut the edge of the first opening, the second rotating blocks of the two adjacent mortise and tenon connectors abut the edge of the second opening, or the third rotating blocks of the two adjacent mortise and tenon connectors abut an edge of the third opening, the two adjacent mortise and tenon connectors stop rotating.

19. The guiding device according to claim 18, wherein a preset angle is formed between an opening direction of the third opening and an opening direction of the second opening.

20. The guiding device according to claim 17, wherein the mortise and tenon connector further comprises a first clamping slot and a second clamping slot respectively formed in opposite sides of the second stop block; a first buckle engageable with the first clamping slot and a second buckle engageable with the second clamping slot are respectively arranged on opposite sides of the second opening;
when the bendable tube section is bent, the first rotating block rotates relatively to the first rotating slot, and the second rotating block rotates relatively to the second rotating slot; and
when the second stop blocks of the two adjacent mortise and tenon connectors abut the edge of the second opening, the two adjacent mortise and tenon connectors stop rotating; when the first buckle engages with the first clamping slot or the second buckle engages with the second clamping slot, a relative rotational position between the two adjacent mortise and tenon connectors is fixed.
